(19) United States
(12) Patent Application Publication
(10) Pub. No.: US 2024/0024295 A1
(43) Pub. Date: Jan. 25, 2024

(54) COMPOSITIONS AND METHODS FOR INHIBITING HAIR GROWTH

(71) Applicant: Elise A. Olsen, Chapel Hill, NC (US)

(72) Inventor: Elise A. Olsen, Chapel Hill, NC (US)

(21) Appl. No.: 18/256,524

(22) PCT Filed: Dec. 10, 2021
(86) PCT No.: PCT/US2021/062777

§ 371 Date: Jun. 8, 2023

Related U.S. Application Data
(60) Provisional application No. 63/124,503, filed on Dec. 11, 2020.

(51) Int. Cl. A61K 31/426; A61K 31/4709; A61K 8/49; A61K 9/00; A61K 9/06; A61K 9/08; A61Q 7/02

(52) U.S. Cl. CPC A61K 31/426; A61K 31/4709; A61K 8/4926; A61K 8/496; A61K 9/0014; A61K 9/06; A61K 9/08; A61Q 7/02

(57) ABSTRACT

A method for inhibiting hair growth in mammals using compositions containing thiazoiidine or quinoline carboxamide FP receptor antagonists. The compositions can be applied topically to the skin and/or hair. The compositions can (1) arrest hirsutism or hypertrichosis, (2) reverse hirsutism, hypertrichosis, and/or unwanted hair, and (3) prevent or limit hair growth including after hair removal by chemical or physical methods. These compositions can also be used to help prevent or to limit the degree of hair loss from chemotherapy/cytotoxic chemical agents or radiation.

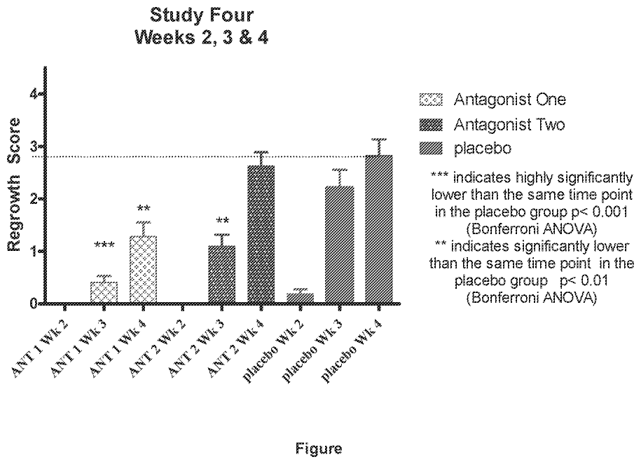

### RELATED APPLICATIONS

This application claims priority to U.S. provisional application No. 63/124,503, filed Dec. 11, 2020, which is incorporated herein by reference in its entirety.

### FIELD OF THE INVENTION

This invention relates to compositions and methods for inhibiting hair growth in mammals. More particularly, this invention relates to compositions and methods for slowing or stopping the growth of hair, or both, for inhibiting hair growth to treat conditions such as hirsutism and hypertrichosis, and for preventing chemotherapy or radiation-induced or related hair loss.

### BACKGROUND OF THE INVENTION

There are two main types of increased hair growth, hirsutism and hypertrichosis. Hirsutism is defined as the presence of excess terminal hair in women only and in anatomic sites where hair growth is under androgen control and considered to be a secondary male characteristic (i.e. primarily beard, moustache, chest, and midline of lower abdomen). Hypertrichosis is defined as hair density or length beyond the accepted limits of normal in a given body area for a particular age, race, and gender, may be in one or many body areas, may be terminal, vellus, or lanugo hair, and is not under androgen control. Terminal hair is defined as hair that is similar in diameter to hair growing on the occipital scalp or eyebrows and usually >40 μm in diameter but its maximum length is body site dependent. Vellus and lanugo hair are of diminished diameter and color compared to terminal hair and do not grow beyond several centimeters in maximum length. Both types of increased hair growth can occur secondary to inherited conditions or can occur secondary to the use of certain medications. Hirsutism can be caused by the use of exogenous androgens or medications that bind to the androgen receptor (some progestins and anabolic steroids, for example) and diffuse hypertrichosis can be caused by the use of certain systemic drugs (minoxidil, diazoxide, cyclosporine, for example). Local hypertrichosis may be caused by the use of certain topical drugs, such as prostaglandin F analogs used for glaucoma and topical minoxidil used for hair growth in male or female pattern baldness. In addition, there are situations where the hair growth is determined to be within normal limits for a given ethnic group but increased in comparison with the general population. In some cases, the excess hair growth can be desirable (i.e. in eyelashes) but in general, increased hair growth, especially in women, is viewed as undesirable and various means are utilized to remove the unwanted hair. There are also situations where the amount of hair is not indicative of hypertrichosis or hirsutism, but the subject finds the amount of hair or the frequency of removal undesirable.

The current means of treating hirsutism, hypertrichosis and excessive or unwanted hair include the physical and chemical means of hair removal including shaving, laser hair removal, electrolysis, depilatories, and waxing. These methods all require repetitive treatments, and none ensure complete and lasting hair removal, particularly in the presence of a continued systemic abnormality that is driving a vellus to terminal transition of hair. Currently, there is one FDA approved medication that slows hair growth: a topical omithine decarboxylase inhibitor Eflornithine (Vaniqa®) that shows significant efficacy in only a limited proportion of women with unwanted facial hair and has not been FDA approved to be safe to use on other, larger body surface areas. There are several systemic agents that slow the transition of vellus to terminal hair growth or cause some miniaturization of terminal hair in women with hirsutism, and these agents include systemic antiandrogens (such as spironolactone, flutamide, and cyproterone acetate) and 5α-reductase inhibitors (such as finasteride or dutasteride). However, these agents do not cause total removal of the unwanted hair nor are they FDA approved for this indication. In addition, these systemic agents all have the risk of feminization of a male fetus in women of child-bearing potential and possibly other side effects as well. All of these methods, topical or systemic, require the continued use of other agents to remove unwanted hair. Thus, there is a need for new products that work in a higher percentage of patients with unwanted hair, including those caused by hypertrichosis or hirsutism, and/or have fewer and less severe side effects than currently available products.

Another unmet need is hair loss related to chemotherapy or radiation [also called chemotherapy induced alopecia (CIA) or radiation-induced hair loss], particularly that occurring in women. Many cytotoxic chemotherapeutic agents or radiation that target rapidly growing cancer cells inadvertently also affect the rapidly growing anagen hair matrix cells, causing a profound and psychologically debilitating hair loss. This hair loss may make women in particular choose alternate therapies that do not cause this adverse effect, and/or it may cause a great deal of depression and/or anxiety during the entire cancer treatment process and beyond, should the hair loss persist. Persistent CIA is now a well-recognized problem for women with breast or ovarian cancer having been treated successfully with certain chemotherapy regimens.

Prostaglandins have been shown in vivo to increase hair length. Naturally occurring prostaglandins (e.g., PGA₂, PGB₂, PGE₁, PGE₂, PGF_{2α}, and PGI₂) are C-20 unsaturated fatty acids. PGF_{2α}, the naturally occurring Prostaglandin F (PGF) analog in humans, is characterized by hydroxyl groups at the C₉ and C₁₈ positions on the alicyclic ring, a cis-double bond between C₅ and C₆, and a trans-double bond between C₁₃ and C₁₄. PGF₂, has the formula:

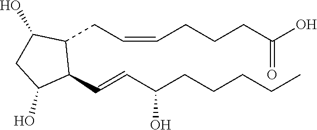 [SMILES: CCCCC[C@H](O)/C=C/[C@@H]1[C@@H](C/C=C\CCCC(=O)O)[C@@H](O)C[C@H]1O]

Analogs of naturally occurring Prostaglandin F are known in the art. For example, see U.S. Pat. No. 4,024,179 issued to Bindra and Johnson on May 17, 1977; German Patent No. DT-002,460,990 issued to Beck, Lerch, Seeger, and Teufel published on Jul. 1, 1976; U.S. Pat. No. 4,128,720 issued to Hayashi, Kori, and Miyake on Dec. 5, 1978; U.S. Pat. No. 4,011,262 issued to Hess, Johnson, Bindra, and Schaaf on Mar. 8, 1977; U.S. Pat. No. 3,776,938 issued to Bergstrom and Sjovall on Dec. 4, 1973; P. W. Collins and S. W. Djuric, “Synthesis of Therapeutically Useful Prostaglandin and Prostacyclin Analogs”, Chem. Rev. Vol. 93 (1993), pp. 1533-1564; G. L. Bundy and F. H. Lincoln, “Synthesis of 17-Phenyl-18,19,20-Trinorprostaglandins: I. The PG Series”, Prostaglandin, Vol. 9 No. 1 (1975), pp. 1-4; W. Bartman, G. Beck, U. Lerch, H. Teufel, and B. Scholkens, “Luteolytic Prostaglandin: Synthesis and Biological Activity”, Prostaglandin, Vol. 17 No. 2 (1979), pp. 301-311; C. Iiljebris, G. Selen, B. Resul, J. Stemschantz, and U. Hacksell, “Derivatives of 17-Phenyl-18, 19,20-trinorprostaglandin F₂α. Isopropyl Ester: Potential Antiglaucoma Agents”, Journal of Medicinal Chemistry, Vol. 38 No. 2 (1995), pp. 289-304.

Prostaglandins in general have a wide range of biological activities. For example, PGE2 has the following properties: a) regulator of cell proliferation, b) regulator of cytokine synthesis, c) regulator of immune responses and d) inducer of vasodilatation. Vasodilatation is thought to be one of the mechanisms of how minoxidil provides a hair growth benefit. In vitro results in the literature also indicate some anti-inflammatory properties of the prostaglandins. c.f.; Tanaka, H. Br J. Pharm. (1995) 116, 2298.

### SUMMARY OF THE INVENTION

In certain embodiments, provided are methods of inhibiting hair growth, the method comprising administering to a subject a safe and effective amount of at least one FP receptor antagonist. Inhibiting hair growth may include slowing hair growth. Inhibiting hair growth may include stopping hair growth. Inhibiting hair growth may include preventing regrowth after hair removal. Inhibiting hair growth may include helping to protect hair from agents (chemotherapy or radiation) that will cause hair loss. The inhibition of hair growth may treat at least one of hirsutism, hypertrichosis, and/or unwanted hair including medication induced unwanted hair, and may help to prevent regrowth after physical or chemical removal of hair or limit chemotherapy induced alopecia, radiation-induced hair loss, and a combination thereof.

The FP receptor antagonist may be a compound of Formula (I), or a pharmaceutically acceptable salt thereof,

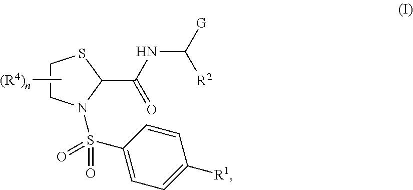

wherein G, R¹, R², R⁴, and n are as described below.

The FP receptor antagonist may be a compound of Formula (II), or a pharmaceutically acceptable salt thereof,

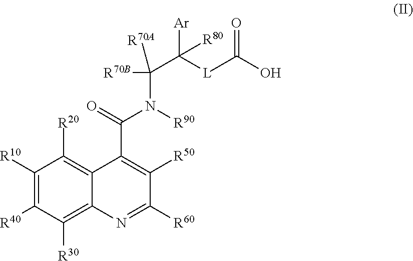

wherein R¹⁰, R²⁰, R³⁰, R⁴⁰, R⁵⁰, R⁶⁰, R^{70A}, R^{70B}, R⁸⁰, R⁹⁰, and Ar are as described below.

Further provided are methods of treating a condition, the method comprising administering to a subject a safe and effective amount of at least one FP receptor antagonist, wherein the condition is selected from at least one of hirsutism, hypertrichosis, unwanted hair, or prevention of regrowth after hair removal. Further provided are methods of preventing or limiting chemotherapy-related hair loss, radiation-related hair loss, and a combination thereof. In certain embodiments, the condition is hirsutism, hypertrichosis, unwanted hair, prevention of regrowth after hair removal, chemotherapy-related hair loss, and/or radiation-related hair loss. The FP receptor antagonist may be a compound of Formula (I) or (II), or a pharmaceutically acceptable salt thereof.

Further provided are methods of preventing or limiting chemotherapy-related hair loss or radiation-related hair loss, the method comprising administering to a subject a safe and effective amount of at least one FP receptor antagonist. The FP receptor antagonist may be administered prior to chemotherapy or radiation, inhibit hair growth, and render the subject less susceptible to chemotherapy-related hair loss or radiation-related hair loss. The FP receptor antagonist may be a compound of Formula (I) or (II), or a pharmaceutically acceptable salt thereof.

Further provided are methods of inhibiting chemotherapy-related hair loss or radiation-related hair loss, the method comprising administering to a subject a safe and effective amount of a FP receptor antagonist. The FP receptor antagonist may be a compound of Formula (I) or (II), or a pharmaceutically acceptable salt thereof.

Further provided are pharmaceutical compositions comprising an FP receptor antagonist, a carrier; and at least one activity enhancer selected from the group consisting of i) hair growth inhibitor, ii) hirsutism treatment agent, iii) preventing or limiting chemotherapy-related hair loss or radiation-related hair loss or regrowth after removal of hair, and iv) penetration enhancer. The FP receptor antagonist may be a compound of Formula (I) or (II), or a pharmaceutically acceptable salt thereof. The activity enhancer may be eflornithine or spironolactone.

### BRIEF DESCRIPTION OF THE FIGURE

The FIGURE shows the effect of two FP receptor antagonists to inhibit hair regrowth compared to placebo.

### DETAILED DESCRIPTION OF THE INVENTION

One object of this invention is to provide methods for using prostaglandin antagonists to inhibit the growth of hair and to provide compositions that inhibit hair growth. It is a further object of the invention to provide a selection of appropriate prostaglandin FP antagonists that will inhibit hair growth and thus treat diseases and conditions marked by increased or unwanted growth of hair, may include preventing or delaying hair regrowth after hair removal by various physical or chemical methods, and/or prevent or limit hair loss that is caused by chemotherapy or radiation induced hair loss. This invention relates to compositions comprising FP receptor antagonists (e.g., prostaglandin F receptor antagonists) to treat hirsutism, hypertrichosis, and unwanted hair including regrowth after removal of unwanted hair, and to prevent chemotherapy-induced or related hair loss, and/or radiation-induced or related hair loss. Treatment includes arresting or slowing hair growth. The prostaglandin antagonist may interact strongly with hair-selective receptors, such as the FP receptor. The prostaglandin analog may selectively inhibit activation of the FP receptor and not activate any other receptors that would negate the effect of inhibiting the FP receptor.

There are multiple ways of inhibiting the function of the FP receptor. A particularly thorough explanation of each of those ways is found in US 2007/0004620 A1, which is specifically incorporated into this application by way of reference, and each of those methods of inhibiting the activation of the FP receptor is specifically contemplated for the current use. To illustrate, some of the more useful methods are described below, but the lack of an illustration is not to be construed as a lack of specific contemplation.

Publications and patents are referred to throughout this disclosure. All U.S. patents and publications cited herein are hereby incorporated by reference.

All percentages, ratios, and proportions used herein are by weight unless otherwise specified.

In the description of the invention various embodiments and individual features are disclosed. As will be apparent to a person having ordinary skill in the art, all combinations of such embodiments and features are possible and can result in preferred embodiments of the invention.

### Definition and Usage of Terms

The following is a list of definitions for terms, as used herein:

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, r-heptyl, n-octyl, n-nonyl, and n-decyl.

The term “alkylene,” as used herein, refers to a divalent group derived from a straight or branched chain hydrocarbon, for example, of 1 to 3 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). A C₃₋₆cycloalkyl is monocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “halogen” or “halo,” as used herein, means Cl, Br, I, or F.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

“Pharmaceutically acceptable” means suitable for use in a human or other mammal.

The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloride, hydrobromide, sulfuric, phosphoric and the like.

“Safe and effective amount” means a quantity of a compound high enough to provide a significant positive modification of the subject's condition to be treated, but low enough to avoid serious side effects (at a reasonable benefit/risk ratio).

### Methods of the Invention

This invention further relates to a method for inhibiting hair growth in mammals. The method comprises administering to a mammal (preferably a human) suffering from excess hair growth, a FP receptor antagonist described herein. For example, a mammal diagnosed with hirsutism can be treated by the methods of this invention. A mammal diagnosed with hypertrichosis can be treated by the methods of this invention. A mammal with unwanted hair can be treated by the methods of this invention. A mammal with any of these conditions may be treated by the methods of this invention to prevent regrowth after physical or chemical removal of unwanted hair Preferably, a systemic or topical composition comprising A) the FP receptor antagonist and B) a carrier is administered to the mammal. More preferably, the composition is a topical composition comprising A) the FP receptor antagonist, B) the carrier, and C) an optional activity enhancer.

This invention further relates to a method for preventing, inhibiting, limiting, or reducing chemotherapy-related or radiation-related hair loss. The methods may comprise administering to a subject (such as a mammal, preferably a human) a FP receptor antagonist described herein. The FP receptor antagonist may be applied topically to the scalp, eyebrows, or eyelashes. The FP receptor antagonist may be applied prior to, during, and/or after chemotherapy or radiation treatment or after physical or chemical hair removal. The FP receptor antagonist may transiently inhibit the proliferation of the hair follicle matrix cells and inhibit hair growth, making the hair less susceptible to the effects of the cytotoxic agent or radiation. This in turn could help to prevent hair loss or slow the amount of hair loss from chemotherapy or radiation therapy. This could not only lead to greater compliance with a chemotherapy or radiation regimen that is a known cause of hair loss, but also would have dramatic effects on the psychological and emotional well-being of patients with cancer going through chemotherapy or radiation.

In the following, embodiments of the invention are disclosed. The first embodiment is denoted E1, the second embodiment is denoted E2 and so forth.

E1. A method of inhibiting hair growth, the method comprising administering to a subject in need thereof, a safe and effective amount of a compound of formula (I), or a pharmaceutically acceptable sat thereof,

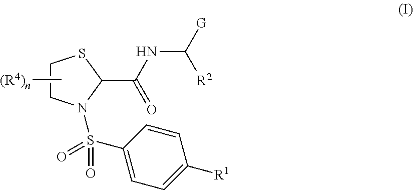

wherein:
G is

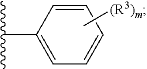

R¹ is tert-butyl or phenyl, wherein the phenyl is optionally substituted with 1-3 R^{1a};
R^{1a}, at each occurrence, is independently C₁₋₄alkyl, C₁₋₄fluoroalkyl, halogen, cyano, —OC₁₋₄ alkyl, or —OC₁₋₄fluoroalkyl;
R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a};
R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄ alkylene-OH, or —C₁₋₄alkylene-OC₁₋₄alkyl;
R³, at each occurrence, is independently C₁₋₄alkyl, C₁₋₄fluoroalkyl, halogen, cyano, —OC₁₋₄ alkyl, or —OC₁₋₄fluoroalkyl;
R⁴, at each occurrence, is independently C₁₋₄alkyl or C₁₋₄fluoroalkyl;
m is 0, 1, 2, 3, 4, or 5; and
n is 0, 1, 2, 3, or 4.

E2. A method of treating a condition, the method comprising administering to a subject in need thereof, a safe and effective amount of a compound of formula (I), or a pharmaceutically acceptable salt thereof, wherein the condition is selected from at least one of hirsutism, hypertrichosis, unwanted hair, chemotherapy-related hair loss, radiation-related hair loss, and a combination thereof,

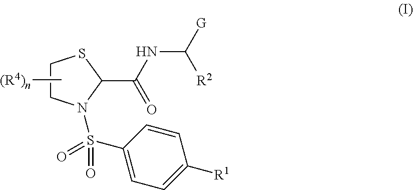

wherein:
G is

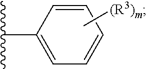

R¹ is tert-butyl or phenyl, wherein the phenyl is optionally substituted with 1-3 R^{1a};
R^{1a}, at each occurrence, is independently C₁₋₄alkyl, C₁₋₄fluoroalkyl, halogen, cyano, —OC₁₋₄ alkyl, or —OC₁₋₄fluoroalkyl;
R² is —C₁₋₄alkylene-OH, —C₁₋₄alkylene-OC₁₋₄alkyl; —C₁₋₄alkylene-OC(O)C₁₋₄alkyl, or —C₁₋₄alkylene-OC(O)CH(NH₂)R^{2a};
R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄ alkylene-OH, or —C₁₋₄alkylene-OC₁₋₄alkyl;
R³, at each occurrence, is independently C₁₋₄alkyl, C₁₋₄fluoroalkyl, halogen, cyano, —OC₁₋₄ alkyl, or —OC₁₋₄fluoroalkyl;
R⁴, at each occurrence, is independently C₁₋₄alkyl or C₁₋₄fluoroalkyl;
m is 0, 1, 2, 3, 4, or 5; and
n is 0, 1, 2, 3, or 4.

E3. The method of any of E1-E2, wherein R¹ is phenyl.

E4. The method of any of E1-E3, wherein R² is —C₁₋₄alkylene-OH.

E5. The method of E4, wherein R² is —CH₂CH₂OH.

E6. The method of any of E1-E3, wherein R² is —C₁₋₄alkylene-OC(O)CH(NH₂)R^{2a}, such as (S)—C₁₋₄alkylene-OC(O)CH(NH₂)R^{2a} or (S)—C₁₋₄alkylene-OC(O)CH(NH₂)R^{2a}. Preferably. —C₁₋₄alkylene-OC(O)CH(NH₂)R^{2a} is (S)—C₁₋₄alkylene-OC(O)CH(NH₂)R^{2a}. Preferred pharmaceutically acceptable salts of the primary amino group include the hydrochloride, mesylate, hydrosulfate, sulfate, dihydrophosphate, citrate, edisylate, and fumarate.

E7. The method of E6, wherein R² is —CH₂CH₂—OC(O)CH(NH₂)R^{2a}, such as (S)—CH₂CH₂—OC(O)CH(NH₂)R^{2a} or (R)—CH₂CH₂—OC(O)CH(NH₂)R^{2a}. Preferably, —CH₂CH₂—OC(O)CH(NH₂)R^{2a} is (S)—CH₂CH₂—OC(O)CH(NH₂)R^{2a}. Preferred pharmaceutically acceptable salts of the primary amino group include the hydrochloride, mesylate, hydrosulfate, sulfate, dihydrophosphate, citrate, edisylate, and fumarate.

E8. The method of E6, wherein R² is —C₁₋₄alkylene-OC(O)CH(NH₂)CH(CH₃)₂, such as (S)—C₁₋₄alkylene-OC(O)CH(NH₂)CH(CH₃)₂ or (R)—C₁₋₄alkylene-OC(O)CH(NH₂)CH(CH₃)₂. Preferably, —C₁₋₄alkylene-OC(O)CH(NH₂)CH(CH₃)₂, is (S)—C₁₋₄ alkylene-OC(O)CH(NH₂)CH(CH₃)₂. Preferred pharmaceutically acceptable salts of the primary amino group include the hydrochloride, mesylate, hydrosulfate, sulfate, dihydrophosphate, citrate, edisylate, and fumarate.

E9. The method of E8, wherein R² is —CH₂CH₂—OC(O)CH(NH₂)CH(CH₃)₂, such as (S)—CH₂CH₂—OC(O)CH(NH₂)CH(CH₃)₂ or (R)—CH₂CH₂—OC(O)CH(NH₂)CH(CH₃)₂. Preferably, —CH₂CH₂—OC(O)CH(NH₂)CH(CH₃)₂ is (S)—CH₂CH₂—OC(O)CH(NH₂)CH(CH₃)₂. Preferred pharmaceutically acceptable salts of the primary amino group include the hydrochloride, mesylate, hydrosulfate, sulfate, dihydrophosphate, citrate, edisylate, and fumarate.

E10. The method of any of E1-E9, wherein R³ is fluoro, chloro, CH₃, or OCH₃; and m is 0, 1, or 2.

E11. The method of any of E1-E10, wherein G is

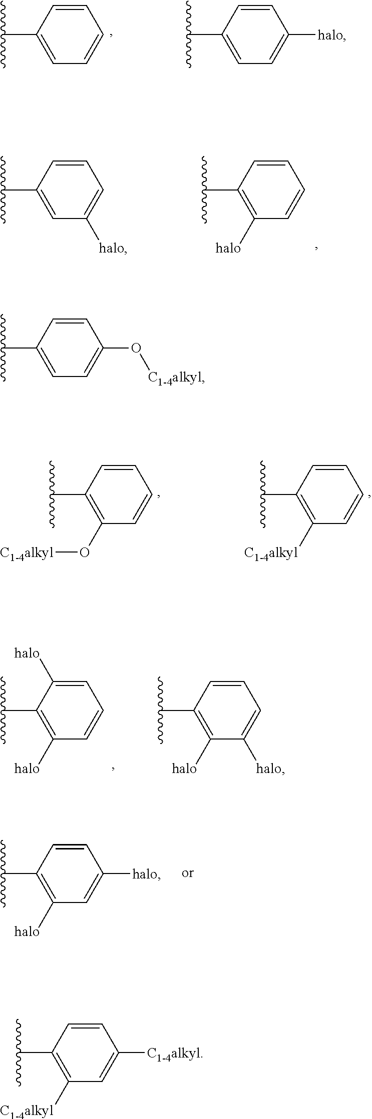

E12. The method of E11, wherein G is

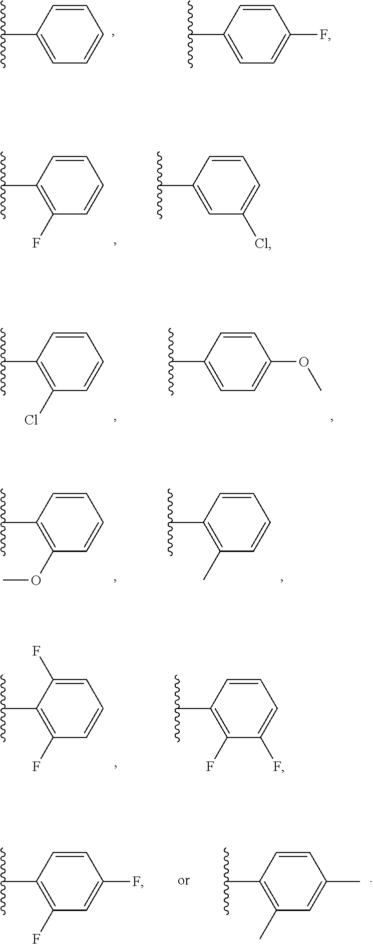

E13. The method of any of E1-E12, wherein n is 0.

E14. The method of any of E1-E13, wherein the compound of formula (I) has formula (I-A)

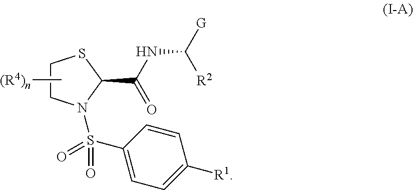

E15. The method of any of E1-E2, wherein the compound of formula (I) is

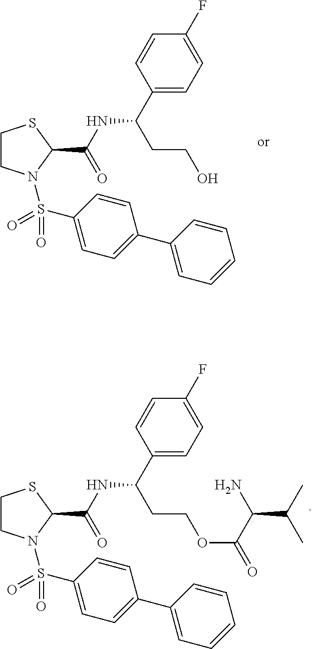 [SMILES: O=C(N[C@@H](CCO)c1ccc(F)cc1)[C@@H]1SCCN1S(=O)(=O)c1ccc(-c2ccccc2)cc1 ; CC(C)[C@H](N)C(=O)OCC[C@H](NC(=O)[C@@H]1SCCN1S(=O)(=O)c1ccc(-c2ccccc2)cc1)c1ccc(F)cc1]

E16. A method of inhibiting hair growth, the method comprising administering to a subject in need thereof, a safe and effective amount of a compound of formula (II), or a pharmaceutically acceptable salt thereof,

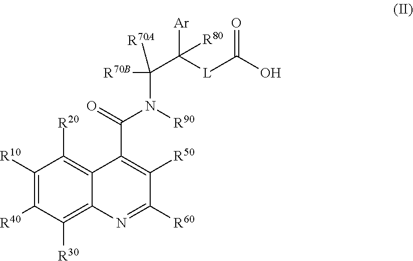

wherein
Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄ alkyl, C₁₋₄cycloalkyl, —OC₁₋₂alkyl, —SC₁₋₂alkyl, C₁₋₄alkyl substituted by 1-3 fluoro, C₃₋₄ cycloalkyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy;
L is a bond or —X—(CR^{a}R^{b})ₖ—;
X is a bond, CH₂, O, S, S(O), S(O)₂, or N(R);
R is hydrogen or CH₃;
R^{a} and R^{b} are independently hydrogen, fluoro, or CH₃; or R^{a} and R^{b} together with the carbon atom to which they attach form a cyclopropyl;
k is 1, 2, 3, or 4;
R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ are independently hydrogen, halogen, CH₃, CH₂F, CHF₂, or CF₃;
R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ is —NR^{c}R^{d};
R^{c} is hydrogen or C₁₋₃alkyl;
R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} are independently hydrogen or CH₃, or R^{70A} and R^{70B} together with the carbon atom to which they attach form a cyclopropyl;
R⁸⁰ is hydrogen, fluoro, CH₃, CF₃, CH₂CH₃, or OH; and
R⁹⁰ is hydrogen or CH₃.

E17. A method of treating a condition, the method comprising administering to a subject in need thereof, a safe and effective amount of a compound of formula (II), or a pharmaceutically acceptable salt thereof, wherein the condition is selected from at least one of hirsutism, hypertrichosis, unwanted hair, chemotherapy-related hair loss, radiation-related hair loss, and a combination thereof,

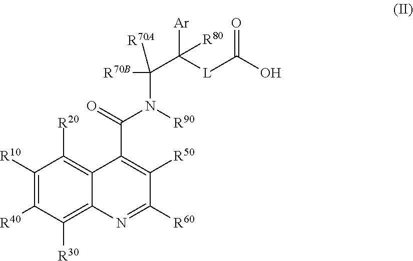

wherein
Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄ alkyl, C₃₋₄cycloalkyl, —OC₁₋₂alkyl, —SC₁₋₂alkyl, C₁₋₄alkyl substituted by 1-3 fluoro, C₃₋₄ cycloalkyl substituted by 1-4 fluoro, —OC₁₋₂alkyl substituted by 1-3 fluoro, and —SC₁₋₂ alkyl substituted by 1-3 fluoro, or where two substituents of the phenyl or pyridinyl on adjacent ring atoms together form a methylenedioxy or ethylenedioxy;
L is a bond or —X—(CR^{a}R^{b})ₖ—;
X is a bond, CH2, O, S, S(O), S(O)₂, or N(R);
R is hydrogen or CH₃;
R^{a} and R^{b} are independently hydrogen, fluoro, or CH₃; or R^{a} and R^{b} together with the carbon atom to which they attach form a cyclopropyl;
k is 1, 2, 3, or 4;
R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ are independently hydrogen, halogen, CH₃, CH₂F, CHF₂, or CF₃;
R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ is —NR^{c}R^{d};
R^{c} is hydrogen or C₁₋₃alkyl;
R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C1-4alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} are independently hydrogen or CH₃, or R^{70A} and R^{70B} together with the carbon atom to which they attach form a cyclopropyl;
R⁸⁰ is hydrogen, fluoro, CH₃, CF₃, CH₂CH₃, or OH: and
R⁹⁰ is hydrogen or CH₃.

E18. The method of E16 or E17, wherein,
Ar is phenyl, wherein the phenyl is unsubstituted or substituted with 1-4 fluoro, or substituted with 1-3 substituents independently selected from the group consisting of fluoro, chloro, CH₃, CHF₂, CF₃, OCH₃, OCHF₂, and OCF₃;
L is a bond or —(CR^{a}R^{b})ₖ—
k is 1, 2, or 3;
R¹⁰ is bromo or ethynyl;
R²⁰, R³⁰, and R⁴⁰ are hydrogen;
R⁵⁰ is chloro or CH₃;
R^{c} is hydrogen or CH₃;
R^{d} is C₁₋₄alkyl, C₁₋₄alkyl substituted by 1-3 fluoro, or C₁₋₄alkyl monosubstituted by phenyl; or
R^{c} and R^{d} together with the nitrogen to which they attach form a saturated or partially unsaturated 5- to 7-membered monocyclic or 7- to 10-membered bicyclic heterocyclic ring, the heterocyclic rings optionally containing a ring member selected from the group consisting of O, N, and S, wherein the heterocyclic rings are optionally substituted with 1-2 substituents independently selected from the group consisting of CH₃, CH₂CH₃, CH(CH₃)₂, CHF₂, CF₃. CH₂CHF₂, and CH₂CF₃, and optionally substituted with 1-4 fluoro; and
R^{70A}, R^{70B}, R⁸⁰, and R⁹⁰ are hydrogen.

E19. The method of E18, wherein,
Ar is phenyl, wherein the phenyl is unsubstituted or substituted with 1-3 substituents independently selected from the group consisting of fluoro, chloro, CH₃, CF₃, OCH₃, OCHF₂, and OCF₃;
L is —CH₂CH₂—
R¹⁰ is bromo;
R⁵⁰ is CH₃;
R⁸⁰ is

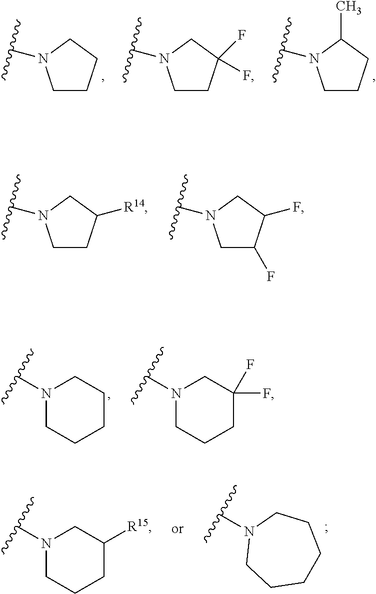

R¹⁴ is fluoro or CH₃; and
R¹⁵ is fluoro, CH₃, or CH₂CH₃.

E20. The method of E19, wherein the compound of formula (II) is

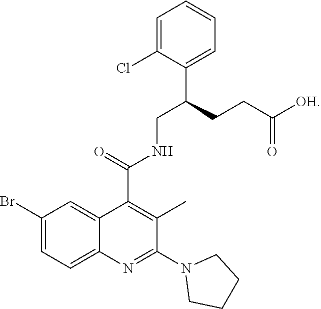 [SMILES: Cc1c(N2CCCC2)nc2ccc(Br)cc2c1C(=O)NC[C@H](CCC(=O)O)c1ccccc1Cl]

E21. The method of any of E1, E3-E16, or E18-E20, wherein inhibiting hair growth includes slowing hair growth.

E22. The method of any of E1, E3-E16, or E18-E20, wherein inhibiting hair growth includes stopping hair growth.

E23. The method of any of E1, E3-E16, or E18-E20, wherein inhibiting hair growth includes preventing hair regrowth after hair removal.

E24. The method of any of E1, E3-E16, or E18-E20, wherein the inhibition of hair growth treats at least one of hirsutism, hypertrichosis, or unwanted hair, and/or prevents or limits at least one of regrowth after hair removal, chemotherapy-related hair loss, or radiation-related hair loss, and a combination thereof.

E25. The method of any of E2-E15 or E17-E20, wherein the condition is hirsutism.

E26. The method of any of E2-E15 or E17-E20, wherein the condition is hypertrichosis.

E27. The method of any of E2-E15 or E17-E20, wherein the condition is unwanted hair.

E28. The method of any of E2-E15 or E17-E20, wherein the condition is chemotherapy-related hair loss.

E29. The method of any of E2-E15 or E17-E20, wherein the condition is radiation-related hair loss.

E30. The method of E28 or E29, wherein the compound of formula (I) or (II), or pharmaceutically acceptable salt thereof, is administered prior to chemotherapy or radiation, and inhibits hair growth and renders the subject less susceptible to chemotherapy-related hair loss or radiation-related hair loss.

Preferred pharmaceutically acceptable salts of the primary amino group include the hydrochloride, mesylate, hydrosulfate, sulfate, dihydrophosphate, citrate, edisylate, and fumarate.

The compounds of formula (I), and salts thereof, corresponding methods of synthesis, and methods of testing for FP receptor antagonist activity are described in U.S. Pat. Nos. 8,415,480, 9,447,055, 9,834,528, and 10,259,795 which are incorporated herein by reference.

The compounds of formula (II), and salts thereof, corresponding methods of synthesis, and methods of testing for FP receptor antagonist activity are described in US2020/0157073, which is incorporated herein by reference.

The dosage of the FP receptor antagonist administered depends on the method of administration. For systemic administration, (e.g., oral, rectal, nasal, sublingual, buccal, or parenteral), typically, 0.5 mg to 300 mg, preferably 0.5 mg to 100 mg, more preferably 0.1 mg to 10 mg, of a FP receptor antagonist described above is administered per day. These dosage ranges are merely exemplary, and daily administration can be adjusted depending on various factors. The specific dosage of the FP receptor antagonist to be administered, as well as the duration of treatment, and whether the treatment is topical or systemic are interdependent. The dosage and treatment regimen will also depend upon such factors as the specific FP receptor antagonist used, the treatment indication, the efficacy of the compound, the personal attributes of the subject (such as, for example, weight, age, sex, and medical condition of the subject), compliance with the treatment regimen, and the presence and severity of any side effects of the treatment.

For topical administration (e.g., local application on the skin, ocular, liposome delivery systems, or iontophoresis), the topical composition is typically administered from once per day up to four times per day. In general, 2-4 weeks is sufficient to observe a noticeable decrease in hair growth.

### Compositions of the Invention

In one aspect, this invention relates to a composition for treating hirsutism, hypertrichosis, or unwanted hair in mammals. Treating any of these types of increased hair growth includes arresting hair growth or reversing the vellus or lanugo to terminal hair growth transformation, suppressing the hair growth rate, or preventing regrowth after hair removal. This invention also relates to a composition for preventing or limiting chemotherapy or radiation induced or related hair loss in mammals. Treatment of this condition includes arresting anagen hair growth to prevent the temporary effect of chemotherapy or radiation on the hair follicle. The composition comprises A) an FP receptor antagonist or a selective modifier of the FP ligand as described herein and B) a carrier. The composition may further comprise C) one or more optional activity enhancers.

Preferably, A) the FP receptor antagonist is an active ingredient formulated into a composition, such as a pharmaceutical or cosmetic composition, administered for treatment or prophylaxis of a condition, including, for example, hirsutism, hypertrichosis, unwanted hair, chemotherapy-related hair loss, and radiation-related hair loss. Standard pharmaceutical formulation techniques are used, such as those disclosed in Remington's Pharmaceutical Sciences. Mack Publishing Company. Easton, PA. (1990).

Component A) can be a PGF analog having the structure of general Formula (I) or (II), as described herein in E1 to E29.

Preferably, A) the FP receptor antagonist is an active ingredient formulated into a composition, such as a pharmaceutical or cosmetic composition, administered for treatment or prophylaxis of hirsutism, hypertrichosis, unwanted hair, hair regrowth after hair removal, chemotherapy-induced or related hair growth, and/or radiation-induced or related hair growth. Standard pharmaceutical formulation techniques are used, such as those disclosed in Remington's Pharmaceutical Sciences. Mack Publishing Company. Easton, PA. (1990).

The composition further comprises component B) a carrier. “Carrier” means one or more compatible substances that are suitable for administration to a mammal. Carrier includes solid or liquid fillers, diluents, hydrotopes, surface-active agents, and encapsulating substances. “Compatible” means that the components of the composition are capable of being commingled with the FP receptor antagonist, and with each other, in a manner such that there is no interaction which would substantially reduce the efficacy of the composition under ordinary use situations. Carriers must be of sufficiently high purity and sufficiently low toxicity to render them suitable for administration to the mammal being treated. The carrier can be inert, or it can possess pharmaceutical benefits, cosmetic benefits, or both.

The choice of carrier for component B) depends on the route by which A) the FP receptor antagonist will be administered and the form of the composition. The composition may be in a variety of forms, suitable, for example, for systemic administration (e.g., oral, rectal, nasal, sublingual, buccal, or parenteral) or topical administration (e.g., local application on the skin, ocular, liposome delivery systems, or iontophoresis). Topical administration is preferred.

Carriers for systemic administration typically comprise one or more ingredients selected from the group consisting of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others.

Component a) is a diluent. Suitable diluents include sugars such as glucose, lactose, dextrose, and sucrose; polyols such as propylene glycol; calcium carbonate; sodium carbonate; cellulose: glycerin; mannitol; and sorbitol.

Component b) is a lubricant. Suitable lubricants are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma.

Component c) is a binder. Suitable binders include polyvinylpyrilidone; magnesium aluminum silicate; starches such as corn starch and potato starch; gelatin; tragacanth; and cellulose and its derivatives, such as sodium carboxymethylcellulose, ethyl cellulose, methylcellulose, and sodium carboxymethylcellulose.

Component d) is a disintegrant. Suitable disintegrants include starches, agar, alginic acid and the sodium salt thereof, effervescent mixtures, and croscarmelose.

Component e) is a colorant such as an FD&C dye.

Component f) is a flavor such as menthol, peppermint, and fruit flavors.

Component g) is a sweetener such as aspartame and saccharin.

Component h) is an antioxidant such as BHA, BHT, and vitamin E.

Component j) is a preservative such as methyl paraben and sodium benzoate.

Component k) is a glidant such as silicon dioxide.

Component m) is a solvent, such as water, isotonic saline, ethyl oleate, alcohols such as ethanol, and phosphate buffer solutions.

Component n) is a suspending agent. Suitable suspending agents include cellulose and its derivatives, such as methyl cellulose and sodium carboxymethyl cellulose; Avicel® RC-591 from FMC Corporation of Philadelphia, Pennsylvania; tragacanth and sodium alginate.

Component o) is a wetting agent such as lecithin, polysorbate 80, and sodium lauryl sulfate.

Component p) is a surfactant such as the TWEENS® from Atlas Powder Company of Wilmington, Delaware.

Compositions for parenteral administration typically comprise A) 0.1 to 10% of a FP receptor antagonist and B) 90 to 99.9% of a carrier comprising a) a diluent, b) a lubricant, c) a binder, and m) a solvent. Preferably, component a) is propylene glycol, b) is sesame oil, c) is pyrrolidone, and m) is ethanol or ethyl oleate.

Compositions for oral administration can have various dosage forms. For example, solid forms include tablets, capsules, granules, and bulk powders. These oral dosage forms comprise a safe and effective amount, usually at least 5%, and preferably from 25% to 50%, of A) the FP receptor antagonist. The oral dosage compositions further comprise B) 50 to 95% of a carrier, preferably 50 to 75%.

Tablets can be compressed, tablet triturates, enteric-coated, sugar-coated, film-coated, or multiple-compressed. Tablets typically comprise A) the FP receptor antagonist, and B) a carrier comprising ingredients selected from the group consisting of a) diluents, b) lubricants, c) binders, d) disintegrants, e) colorants, f) flavors, g) sweeteners, k) glidants, and combinations thereof. Preferred diluents include calcium carbonate, sodium carbonate, mannitol, lactose and cellulose. Preferred binders include starch, gelatin, and sucrose. Preferred disintegrants include starch, alginic acid, and croscarmellose. Preferred lubricants include magnesium stearate, stearic acid, and talc. Preferred colorants are the FD&C dyes, which can be added for appearance. Chewable tablets preferably contain g) sweeteners such as aspartame and saccharin, or f) flavors such as menthol, peppermint, and fruit flavors.

Capsules (including time release and sustained release formulations) typically comprise A) the FP receptor antagonist, and B) a carrier comprising one or more a) diluents disclosed above in a capsule comprising gelatin. Granules typically comprise A) the FP receptor antagonist, and preferably further comprise k) glidants such as silicon dioxide to improve flow characteristics.

The selection of ingredients in the carrier for oral compositions depends on secondary considerations like taste, cost, and shelf stability, which are not critical for the purposes of this invention. One skilled in the art would know how to select appropriate ingredients without undue experimentation.

The solid compositions may also be coated by conventional methods, typically with pH or time-dependent coatings, such that A) the FP receptor antagonist is released in the gastrointestinal tract in the vicinity of the desired application, or at various times to extend the desired action. The coatings typically comprise one or more components selected from the group consisting of cellulose acetate phthalate, polyvinylacetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise A) the FP receptor antagonist and B) a carrier comprising ingredients selected from the group consisting of a) diluents, e) colorants, and f) flavors, g) sweeteners, j) preservatives, m) solvents, n) suspending agents, and p) surfactants. Peroral liquid compositions preferably comprise one or more ingredients selected from the group consisting of e) colorants, f) flavors, and g) sweeteners.

Other compositions useful for attaining systemic delivery of the subject compounds include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methyl cellulose. Such compositions may further comprise b) lubricants, e) colorants, f) flavors, g) sweeteners, h) antioxidants, and k) glidants.

The compositions may further comprise component C) an optional activity enhancer. Component C) may be selected from the group consisting of i) hair growth inhibitors (other than the PGF antagonists), ii) hirsutism treatment agents, iii) preventatives of chemotherapy- or radiation-induced or related alopecia or hair loss, and iv) penetration enhancers, or combinations thereof. Components i)-iii) are exemplified by compounds that work marginally, if at all by itself, but can help the activity of the FP antagonist.

In certain embodiments, component C) is a i) hair growth inhibitor. Suitable hair growth inhibitors may include the following: advanced glycation end products (AGE's) and compounds such as lysine (WO2010063678A2, WO2010063673A2); extracts of tetraselmis species (US20100143267A1); fibroblast growth factor (FGF)18 (WO2008102782A1); cytotoxic lectin (US20080145330A1); trypsin and other enzymes (US20070269418A1); extract of Juniperus genus and/or malt extract (U.S. Pat. Nos. 6,375,948 and 7,211,278); hair growth-inhibiting active substances (WO06125582A1); extract of ginger root (US20060099280A1); toxalbumins such as ricin, abrin, or modeccin and the like (US20060034952A1); inhibitors of cysteine pathway enzymes (WO9524885A1); inhibitors of nitric oxide synthetase (WO9524884A1); omithine amino transferase inhibitors (WO9524181A1); cyclooxygenase inhibitors such as NSAIDs (WO9427586A1); 5-lipoxygenase inhibitors (WO9427563A1) substances such as substituted guanidines or amidines (WO8808295A1); and aminobenzophenones (U.S. Pat. No. 3,426,137).

In certain embodiments, the hair growth inhibitor is eflornithine (Vaniqa®).

In certain embodiments, component C) is a ii) hirsutism treatment agent. Suitable hirsutism treatment agents may include the following: botulinum toxin (U.S. Pat. No. 7,754,253); spironolactone (WO9936030A3, WO8700427A1): 2-phenyl-benzothiophene derivatives (U.S. Pat. No. 5,686,468); cyproterone acetate, flutamide, bicalutamide, and inhibitors of 5-alpha reductase such as finasteride dutasteride (U.S. Pat. Nos. 7,744,935, 7,737,288, 7,727,980); N,N-diethy-4-methyl-3-oxo-4-aza-5α-androstane-17β-carboxamide (4-MA, WO9906050A1); PTHR1 receptor ligands (WO2010053548A2); ketaconazole; estrogen receptor modulators such as oral contraceptives (U.S. Pat. No. 5,770,226); progesterone; estrogen (US20070105827); RU58841; neuropeptide Y receptor antagonists; thiazolidinedione derivatives such as rosiglitazone or pioglitazone (U.S. Pat. No. 5,972,944); biguanide (metformin) derivatives; cyoctol [6-(5-methoxy-1-heptyl)-bicyclo (3,3,0)octan-3-one]; botanicals including extracts of Serenoa repens (WO9833472A1); Epilobium species; Cucurbita pepo (U.S. Pat. No. 7,595,346); Urtica dioica; Calluna vulgaris; Populus species; Barosma species; and physical means of hair removal such as laser, electrolysis, and depilatory compounds.

In certain embodiments, the hirsutism treatment agent is oral spironolactone.

In certain embodiments, component C) is a iii) preventative of chemotherapy- or radiation-induced alopecia or hair loss. Suitable preventatives of chemotherapy- or radiation-induced alopecia or hair loss may include the following: 4-((cyanoimino((1,2,2-trimethylpropyl) amino)methyl)amino) benzonitrile (U.S. Pat. No. 6,458,835); growth factors including keratinocyte growth factor, epidermal growth factor, and fibroblast growth factor; prostaglandins including PGE2 and Misoprostol (U.S. Pat. Nos. 7,407,987, 7,388,029); ImuVert; AS101; IL-1; cyclin dependent kinases; p53 inhibitors; capase-3 inhibitors; acylated amino acids including N-acyl cysteine (US20060211659A9); nuclear hormone receptor ligands such as parathyroid hormone antagonist; vitamin and vitamin derivatives such as alpha-tocopherol; M50054; immunosuppressant agents especially cyclosporine; thermal treatments such as scalp hypothermia; angiotensin receptor blockers (US20060135422A1); and oral or topical minoxidil. For example, topical minoxidil has been shown to enhance regrowth after chemotherapy induced alopecia.

In certain embodiments, component C) is a iv) penetration enhancer that can be added to all of the compositions for systemic administration except compositions for oral administration. The amount of component v), when present in the composition, is typically 1 to 5%. Examples of penetration enhancers include 2-methyl propan-2-ol, propan-2-ol, ethyl-2-hydroxypropanoate, hexan-2,5-diol, POE(2) ethyl ether, di(2-hydroxypropyl) ether, pentan-2,4-diol, acetone. POE(2) methyl ether, 2-hydroxypropionic acid, 2-hydroxyoctanoic acid, propan-1-ol, 1,4-dioxane, tetrahydrofuran, butan-1,4-diol, propylene glycol dipelargonate, polyoxypropylene 15 stearyl ether, octyl alcohol, POE ester of oleyl alcohol, oleyl alcohol, lauryl alcohol, dioctyl adipate, dicapryl adipate, di-isopropyl adipate, di-isopropyl sebacate, dibutyl sebacate, diethyl sebacate, dimethyl sebacate, dioctyl sebacate, dibutyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, iso-propyl palmitate, ethyl laurate, 2-ethyl-hexyl pelargonate, iso-propyl isostearate, butyl laurate, benzyl benzoate, butyl benzoate, hexyl laurate, ethyl caprate, ethyl caprylate, butyl stearate, benzyl salicylate, 2-hydroxypropanoic acid, 2-hyroxyoctanoic acid, dimethyl sulphoxide, N,N-dimethyl acetamide, N,N-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacyloheptan-2-one, and combinations thereof.

In certain embodiments of the invention, the FP receptor antagonists are topically administered. Topical compositions that can be applied locally to the skin may be in any form including solutions, oils, creams, ointments, gels, lotions, shampoos, leave-on and rinse-out hair conditioners, milks, cleansers, moisturizers, mousses, sprays, foam, skin patches, and the like. Topical compositions may comprise: component A) the FP receptor antagonists described above and component B) a carrier. The carrier of the topical composition may aid penetration of the FP receptor antagonists into the skin to reach the environment of the hair follicle. Component B) may further comprise one or more optional components. Topical compositions preferably further comprise C) one or more of the optional activity enhancers described above.

The exact amounts of each component in the topical composition depend on various factors. The amount of component A) depends on the binding affinity (IC50) of the FP receptor antagonist selected. The amount of component A) added to the topical composition is:

IC₅₀×10⁻¹≥% of component A)≥IC₅₀×10⁻⁵,

where IC₅₀ is expressed in nanomolar. For example, if the binding affinity of the FP receptor antagonist is 1 nM, the amount of component (A) will be 0.00001 to 0.1%. If the binding affinity of the FP receptor antagonist is 10 nM, the amount of component (A) will be 0.0001 to 0.1%. If the binding affinity of the FP receptor antagonist is 100 nM, the amount of component (A) will be 0.001 to 1.0%. If the binding affinity of the FP receptor antagonist is 1000 nM, the amount of component (A) will be 0.01 to 10%, preferably 0.1 to 5%. The amount and dosage of component A) are critical. If the amount of component A) is outside the ranges specified above (i.e., either higher or lower), efficacy of the treatment will be reduced. In the case of prodrugs such as the amide or ester of an FP receptor antagonist, the IC₅₀ of the free acid, or active form of the drug is to be used to determine its activity.

Component B) the carrier may comprise a single component or a combination of two or more components. Typical carriers for component B) in the topical compositions include water, alcohols, aloe vera gel, allantoin, glycerin, vitamin A and E oils, mineral oil, propylene glycol, PPG-2 myristyl propionate, dimethyl isosorbide, combinations thereof, and the like. Preferred carriers include propylene glycol, dimethyl isosorbide, and water.

The carrier of the topical composition may further comprise one or more ingredients selected from the group consisting of (q) emollients, (r) propellants, (s) solvents, (t) humectants, (u) thickeners, (v) powders, and (w) fragrances.

Ingredient (q) is an emollient. The amount of ingredient (q) in the topical composition is typically 5 to 95%. Suitable emollients include stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, polydimethylsiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Preferred emollients include stearyl alcohol and polydimethylsiloxane.

Ingredient (r) is a propellant. The amount of ingredient (r) in the topical composition is typically 5 to 95%. Suitable propellants include propane, butane, iso-butane, dimethyl ether, carbon dioxide, nitrous oxide, and combinations thereof.

Ingredient (s) is a solvent. The amount of ingredient (s) in the topical composition is typically 5 to 95%. Suitable solvents include water, ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran, and combinations thereof. Preferred solvents include ethyl alcohol.

Ingredient (t) is a humectant. The amount of ingredient (t) in the topical composition is typically 5 to 95%. Suitable humectants include glycerin, sorbitol, sodium 2-pyrrolidone-5-carboxylate, soluble collagen, dibutyl phthalate, gelatin, and combinations thereof. Preferred humectants include glycerin.

Ingredient (u) is a thickener. The amount of ingredient (u) in the topical composition is typically 0 to 95%.

Ingredient (v) is a powder. The amount of ingredient (v) in the topical composition is typically 0 to 95%. Suitable powders include chalk, talc, fullers earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate, and combinations thereof.

Ingredient (w) is a fragrance. The amount of ingredient (w) in the topical composition is typically 0.001 to 0.5%, preferably 0.001 to 0.1%.

Component C) may be an activity enhancer is as described above. Any of the activity enhancers may be added to the topical compositions. In certain embodiments, the topical composition comprises 0.01 to 15% of at least one of components i)-iii). More preferably, the composition comprises 0.1 to 10%, and most preferably 0.5 to 5% of at least one of components i)-iii). In certain embodiments, the topical composition comprises 1 to 5% of component iv).

In certain embodiments, pharmaceutical compositions may further comprise additional active agents including, but not limited to, sunscreens and sunblocks, anti-oxidants/radical scavengers, topical steroids, and retinoids.

In an alternative embodiment of the invention, pharmaceutical compositions for topical administration are prepared by conventional methods. Pharmaceutical compositions for topical administration typically comprise A) a FP receptor antagonist, B) a carrier, such as purified water, and one or more ingredients selected from the group consisting of (y) sugars such as dextrans, particularly dextran 70, (z) cellulose or a derivative thereof, (aa) a salt, (bb) disodium EDTA (Edetate disodium), and (cc) a pH adjusting additive.

Examples of (z) cellulose derivatives suitable for use in the pharmaceutical composition for topical administration include sodium carboxymethyl cellulose, ethyl cellulose, methyl cellulose, and hydroxypropylmethylcellulose. Hydroxypropylmethylcellulose is preferred.

Examples of (aa) salts suitable for use in the for use in the pharmaceutical composition for topical administration include sodium chloride, potassium chloride, and combinations thereof.

Examples of (cc) pH adjusting additives include HCl or NaOH in amounts sufficient to adjust the pH of the pharmaceutical composition for topical administration to 5.2-7.5.

The FP receptor antagonists may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. A preferred formulation for topical delivery of the present compounds uses liposomes as described in Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Wallach and Philippot, “New Type of Lipid Vesicle: Novasome®”, Liposome Technology, Vol. 1, pp. 141-156 (1993); Wallach, U.S. Pat. No. 4,911,928, assigned to Micro-Pak, Inc., issued Mar. 27, 1990; and Weiner et al., U.S. Pat. No. 5,834,014, assigned to The University of Michigan and Micro-Pak, Inc., issued Nov. 10, 1998 (with respect to Weiner et al., with a compound as described herein administered in lieu of, or in addition to, minoxidil).

The FP receptor antagonists may also be administered by iontophoresis. See, e.g., internet site www.unipr.it/arpa/dipfarm/erasmus/erasm14.html; Banga et al., “Hydrogel-based Iontotherapeutic Delivery Devices for Transdermal Delivery of Peptide/Protein Drugs”, Pharm. Res., Vol. 10 (5), pp. 697-702 (1993); Ferry, “Theoretical Model of Iontophoresis Utilized in Transdermal Drug Delivery”, Pharmaceutical Acts Helvetiae, Vol 70, pp. 279-287 (1995); Gangarosa et al., “Modem Iontophoresis for Local Drug Delivery”, Int. J. Pharm, Vol. 123, pp. 159-171 (1995); Green et al., “Iontophoretic Delivery of a Series of Tripeptides Across the Skin in vitro”, Pharm. Res., Vol 8, pp. 1121-1127 (1991); Jadoul et al., “Quantification and Localization of Fentanyl and TRH Delivered by Iontophoresis in the Skin”, Int. J. Pharm., Vol. 120, pp. 221-8 (1995); O'Brien et al., “An Updated Review of its Antiviral Activity, Pharmacokinetic Properties and Therapeutic Efficacy”, Drugs, Vol. 37, pp. 233-309 (1989); Parry et al., “Acyclovir Bioavailability in Human Skin”, J. Invest. Dermatol., Vol. 98 (6), pp. 856-63 (1992); Santi et al., “Drug Reservoir Composition and Transport of Salmon Calcitonin in Transdermal Iontophoresis”, Pharm. Res., Vol 14 (1), pp. 63-6 (1997): Santi et al., “Reverse Iontophoresis—Parameters Determining Electroosmotic Flow: I. pH and Ionic Strength”, J. Control. Release. Vol. 38, pp. 159-165 (1996); Santi et al., “Reverse Iontophoresis—Parameters Determining Electroosmotic Flow: II. Electrode Chamber Formulation”, J. Control. Release, Vol. 42, pp. 29-36 (1996); Rao et al., “Reverse Iontophoresis: Noninvasive Glucose Monitoring in vivo in Humans”, Pharm. Res., Vol. 12 (12), pp. 1869-1873 (1995); Thysman et al., “Human Calcitonin Delivery in Rats by Iontophoresis”, J. Pharm. Pharmacol., Vol. 46, pp. 725-730 (1994); and Volpato et al., “Iontophoresis Enhances the Transport of Acyclovir through Nude Mouse Skin by Electrorepulsion and Electroosmosis”, Pharm. Res., Vol. 12 (11), pp. 1623-1627 (1995).

The FP receptor antagonists may be included in kits comprising a FP receptor antagonist, a systemic or topical composition described above, or both; and information, instructions, or both that use of the kit will provide treatment for conditions including, for example, hirsutism, hypertrichosis, unwanted hair, chemotherapy-related hair loss, and radiation-related hair loss in mammals (particularly humans). The information and instructions may be in the form of words, pictures, or both, and the like. In addition, or in the alternative, the kit may comprise a FP receptor antagonist, a composition, or both; and information, instructions, or both, regarding methods of application of the FP receptor antagonist or composition, preferably with the benefit of inhibiting hair growth in mammals.

In all of the foregoing compositions, and for all routes of administration, the FP receptor antagonists can be used alone or in combinations of two or more FP receptor antagonists. The compositions may further comprise additional drugs or excipients as appropriate for the indication.

### EXAMPLES

These examples are intended to illustrate the invention to those skilled in the art and should not be interpreted as limiting the scope of the invention set forth in the claims.

### Reference Example 1: Analytical Methods

FP receptor antagonists are tested for their potential to grow hair using the Telogen Conversion Assay. The Telogen Conversion Assay measures the potential of a FP receptor antagonist to inhibit in mice the conversion of hair in the resting stage of the hair growth cycle (“telogen”), to the growth stage of the hair growth cycle (“anagen”), and to assess the rate of anagen growth.

Without intending to be limited by theory, there are three principal phases of the hair growth cycle: anagen, catagen, and telogen. It is believed that there is a long telogen period in C3H mice (Harian Sprague Dawley, Inc., Indianapolis, IN) from approximately 40 days of age until about 75 days of age, when hair growth is synchronized. It is believed that after 75 days of age, hair growth is no longer synchronized. Wherein about 40 day-old mice with dark fur (brown or black) are used in hair growth experiments, melanogenesis occurs along with hair (fur) growth wherein the topical application of hair growth inducers is evaluated. The Telogen Conversion Assay herein is used to screen FP receptor antagonists for potential hair growth by measuring melanogenesis and/or inhibition of expected hair growth.

Three groups of 44 day-old C3H mice are used: a vehicle control group, a positive control group, and a test FP receptor antagonist group, wherein the test FP receptor antagonist group is administered a FP receptor antagonist. The length of the assay is typically 24 days with 15 treatment days (wherein the treatment days occur Mondays through Fridays). Day 1 is the first day of treatment. A typical study design is shown in Table 1 below. Typical dosage concentrations are set forth in Table 1, however the skilled artisan will readily understand that such concentrations may be modified.

TABLE 1
Assay Parameters

| | | | Concen- | Application | Length |
|---|---|---|---|---|---|
| Group # | Animal # | Compound | tration | volume | of Study |
| 1 | 1-10 | Test | 0.01% in | 400 μL | 26 days |
| | | Compound | vehicle** | topical | |
| 2 | 11-20 | Positive | 0.01% in | 400 μL | 26 days |
| | | Control | vehicle** | topical | |
| | | (T3)* | | | |
| 3 | 21-30 | Vehicle** | N/A | 400 μL | 26 days |
| | | | | topical | |

*T3 is thyronine
**The vehicle is 60% ethanol, 20% propylene glycol, and 20% dimethyl isosorbide (commercially available from Sigma Chemical Co., St. Louis, MO).

The mice are treated topically Monday through Friday on their lower back (base of tail to the lower rib). A pipette and tip are used to deliver 400 μL to each mouse's back. The 400 μL application is applied slowly while moving hair on the mouse to allow the application to reach the skin.

While each treatment is being applied to the mouse topically, a visual grade of from 0 to 4 will be given to the skin color in the application area of each animal. As a mouse converts from telogen to anagen, its skin color will become less pink and more bluish-black. As indicated in Table 2, the grades 0 to 4 represent the following visual observations as the skin progresses from pink to bluish-black.

TABLE 2
Evaluation Criteria

| Visual Observation | Grade |
|---|---|
| Pink Skin Color | 0 |
| Skin is light gray (indication of initiation of anagen) | 1 |
| Appearance of hair | 2 |
| Spots are aggregating to form one large haired area | 3 |
| Skin almost black with hair covering majority of treatment | 4 |
| area (indication of mouse in full anagen) | |

### Example 1

An FP receptor antagonist having the structure:

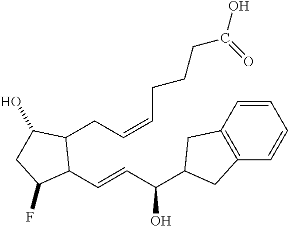 [SMILES: O=C(O)CCC/C=C\CC1C(/C=C/[C@H](O)C2Cc3ccccc3C2)[C@@H](F)C[C@@H]1O]

was tested according to the method of Reference Example 1. The average grade was calculated by averaging the grades of 7 mice after 23 days, 25 days, and 26 days. The results are in Table 3.

### Example 2

An FP receptor antagonist having the structure:

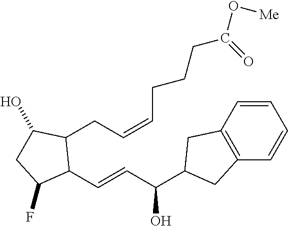 [SMILES: COC(=O)CCC/C=C\CC1C(/C=C/[C@H](O)C2Cc3ccccc3C2)[C@@H](F)C[C@@H]1O]

was tested according to the method of Reference Example 1. The average grade was calculated by averaging the grades of 7 mice after 23 days. The results are in Table 3.

TABLE 3
Average Grades

| Example | 23 Days | 25 Days | 26 Days |
|---|---|---|---|
| 1 | 0.4 | 0.1 | 0.7 |
| 2 | 0.1 | not measured | not measured |

### Example 3

An FP receptor antagonist having the structure:

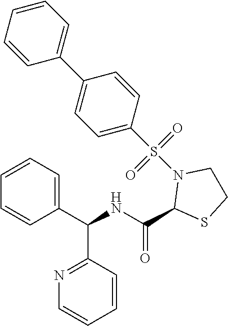 [SMILES: O=C(N[C@H](c1ccccc1)c1ccccn1)[C@@H]1SCCN1S(=O)(=O)c1ccc(-c2ccccc2)cc1]

is tested according to the method of Reference Example 1. The average grade is calculated by averaging the grades of 7 mice after 23 days. The results show that the compound significantly reduces hair growth.

### Example 4

An FP receptor antagonist having the structure:

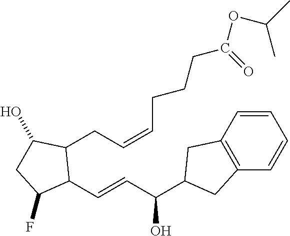 [SMILES: CC(C)OC(=O)CCC/C=C\CC1C(/C=C/[C@H](O)C2Cc3ccccc3C2)[C@@H](F)C[C@@H]1O]

is tested according to the method of Reference Example 1. The average grade is calculated by averaging the grades of 7 mice after 23 days. The results show that the compound significantly reduces hair growth.

### Example 5

An FP receptor antagonist comprising the peptide:
Ile-Leu-Gly-His-(citrulline)-Asp-Tyr-Lys (SEQ ID NO:1)
   is tested according to the method of Reference Example 1. The average grade is calculated by averaging the grades of 7 mice after 23 days. The results show that the compound significantly reduces hair growth.

### Example 6

Two FP receptor antagonists according to the invention (ANT 1, ANT 2) or placebo were applied daily to mice using the Telogen Conversion Assay, as described in Reference Example 1, to assess their ability to slow hair regrowth. ANT 1 is the compound of Example 2, and ANT 2 is the compound of Example 3. In this model, placebo-treated mice began to regrow hair by week two, with significant regrowth by week 4. As shown in the FIGURE, both ANT 1 and ANT 2 at the appropriate dose-level, inhibited hair regrowth at week 3 and week 4 as compared to the control group.

### Example 7

Compositions for topical administration are made, comprising:

| Component | 3-1 | 3-2 | 3-3 | 3-4 |
|---|---|---|---|---|
| PGF antagonist (wt %) | 0.001 | 0.01 | 0.1 | 1.0 |
| IC₅₀ of the FP receptor | 1 | 10 | 100 | 1000 |
| antagonist (nM) | | | | |
| Ethanol (wt %) | 59.99 | 59.9 | 59.4 | 54.0 |
| Propylene Glycol (wt %) | 20.00 | 20.0 | 19.8 | 18.0 |
| Dimethyl Isosorbide (wt | 20.00 | 20.0 | 19.8 | 18.0 |
| %) | | | | |

In the methods of the invention, a subject may be treated with the above composition. Specifically, for 16 weeks, one of the above compositions is daily administered topically to the subject.

### Example 8

A composition for topical administration is made according to the method of Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993), using a PGF analog that is an antagonist of the FP receptor in lieu of cyclosporin A and using the NOVASOME® 1 (available from Micro-Pak, Inc. of Wilmington, Delaware) for the non-ionic liposomal formulation. In the methods of the invention, a subject may be treated with the above composition. Specifically, for 16 weeks, the above composition is administered topically to the subject.

### Example 9

Shampoos or body washes are made, comprising:

| Component | Ex. 6-1 | Ex. 6-2 | Ex. 6-3 | Ex. 6-4 |
|---|---|---|---|---|
| Ammonium Lauryl Sulfate | 11.5% | 11.5% | 9.5% | 7.5% |
| Ammonium Laureth Sulfate | 4% | 3% | 2% | 2% |
| Cocamide MEA | 2% | 2% | 2% | 2% |
| Ethylene Glycol Distearate | 2% | 2% | 2% | 2% |
| Cetyl Alcohol | 2% | 2% | 2% | 2% |
| Stearyl Alcohol | 1.2% | 1.2% | 1.2% | 1.2% |
| Glycerin | 1% | 1% | 1% | 1% |
| Polyquaternium 10 | 0.5% | 0.25% | — | — |
| Polyquaternium 24 | — | — | 0.5% | 0.25% |
| Sodium Chloride | 0.1% | 0.1% | 0.1% | 0.1% |
| Sucrose Polyesters of | 3% | 3% | — | — |
| Cottonate Fatty Acid | | | | |
| Sucrose Polyesters of | 2% | 3% | — | — |
| Behenate Fatty Acid | | | | |
| Polydimethyl Siloxane | — | — | 3% | 2% |
| Cocaminopropyl Betaine | — | 1% | 3% | 3% |
| Lauryl Dimethyl Amine | 1.5% | 1.5% | 1.5% | 1.5% |
| Oxide | | | | |
| Decyl Polyglucose | — | — | 1% | 1% |
| DMDM Hydantoin | 0.15% | 0.15% | 0.15% | 0.15% |
| PGF antagonist having | — | 0.2% | 0.2% | — |
| IC₅₀ of 10 nM | | | | |
| PGF antagonist having | 1.0% | — | — | 1.0 |
| IC₅₀ of 100 nM | | | | |
| Vaniqa | D | | 3% | 2% |
| Phenoxyethanol | 0.5% | 0.5% | 0.5% | 0.5% |
| Fragrance | 0.5% | 0.5% | 0.5% | 0.5% |
| Water | q.s. | q.s. | q.s. | q.s. |

In the methods of the invention, a subject may be treated with the above composition. Specifically, for 12 weeks, a shampoo or body-wash selected from the ones described above is used daily by the subject.

### Example 10

Body washes are made according to Example 9. A human subject suffering from hypertrichosis is treated by a method of this invention. Specifically, for 12 weeks, a body-wash selected from the ones described above is used daily by the subject. A body-wash selected from the ones described above may be used by the subject daily for 12 weeks after physical or chemical hair removal to decrease the growth rate or growth of remaining hairs.

### Example 11

Shampoos are made according to Example 9. A human subject who may be soon to receive or be exposed to agents causing chemotherapy-related or radiation-related hair loss may be treated by a method of this invention. Specifically, for days to several weeks prior to chemotherapy or radiation through days to weeks after chemotherapy or radiation, a shampoo selected from the ones described above is used daily by the subject by applying to the scalp. Solution may also be applied to the eyebrows or eyelashes. The treatment reduces hair loss after chemotherapy or radiation treatment.

### Example 12

Body washes are made according to Example 9. A human subject suffering from unwanted hair is treated by a method of this invention. Specifically, for 12 weeks, a body-wash selected from the ones described above is used daily by the subject. A body-wash selected from the ones described above may be used by the subject daily for 12 weeks either alone or after physical or chemical laser hair removal to decrease the growth rate or growth of remaining hairs.

### Example 13

Methods for testing the prevention of chemotherapy-related alopecia are as follows:
1. Rats are treated with cytosine arabinoside and doxorubicin plus or minus the agent in question prior to injection of chloroleukemic cells.
2. Topical agent in question is applied to anagen test sites in B6D2F1 mice 2 hours prior to intraperitoneal doxorubicin.
3. C57BL/6 mice are treated with agent in question prior to cyclophosphamide.
4. Neonatal rat model is treated with topical application of agent in question prior to either etoposide or cytoxan-doxorubicin combination.

The method for testing the prevention of radiation-induced alopecia is as follows: Mice with hair either synchronously in telogen or anagen (the latter induced by plucking) are pretreated for 3 days with the agent in question prior to irradiation with 10-20 cGy and hair growth is compared to mice irradiated and not treated and non-irradiated mice.

### Example 14: Assays to Determine an FP Antagonist

The FP receptor is a well-described GPCR of 7-transmembrane domains. Assays to determine if a compound is an FP receptor agonist, antagonist, or not are well-known in the art. Examples are cited herein for illustrative purposes only and are not intended to be limiting. Both in vitro and in vivo assays are readily available. One standard in vivo assay is PanLabs' murine antinidatory assay in which pregnant mice are injected with the compound to be tested and then increasing amounts of the known murine-abortafacient, PGF₂ₐ, are injected and an EC₅₀ of protection from the effect of PGF₂ₐ is calculated using a program such as GraphPad Prism.

Several in vitro assays are available (Sharif, et al., Antagonists of FP Prostanoid Receptor-mediated Inositol Phosphates Generation: Comparison with Some Purported FP Antagonists Journal of Pharmacy and Pharmacology Volume 52 Issue 12, Pages 1529-1539, incorporated herein by reference).

Agonist/antagonist assays are available from Ricerca Biosciences (https://pharmacology.ricerca.com/Catalog/Products/ProductDetails.aspx?prodid=HFqdj%2b 3FwaA%3d&path=289&leaf=289&track=Add%2f2%2fTissue+Distribution%2fReproductive).

A mouse preterm parturition model has been recently described that allows for the evaluation of the antagonist potential of FP receptor antagonists (Chollet, et al., BMC Pregnancy and Childbirth 2007, 7 (Suppl 1): S16, incorporated herein by reference in its entirety). Briefly, pregnant CD1 mice at day 14 or 17 are treated with sc RU486 at 2.5 mg/kg in 5 mL. The animals are then treated (orally, sc, im, or ip) with the putative agonist, and its ability to prevent preterm parturition is measured.
1. Jimenez J J, Roberts S M, Mejia J, Mauro L M, Munson J W, Elgart G W, et al. Prevention of chemotherapy-induced alopecia in rodent models. Cell Stress Chaperones 2008; 13(1):31-38.
2. Hussein A M, Jimenez J J, McCall C A, Yunis A A. Protection from chemotherapy-induced alopecia in a rat model. Science 1990; 249(4976): 1564-6.
3. Sredni B, Xu R H, Albeck M, Gafter U. Gal R, Shani A, et al. The protective role of the immunomodulator AS101 against chemotherapy-induced alopecia studies on human and animal models. Int J Cancer 1996; 65(1):97-103.
4. Jimenez J J, Wong G H W, Yunis A A. Interleukin 1 protects from cytosine arabinoside-induced alopecia in a rat model. FASEB J 1991; 5(10):2456-2458.
5. Hussein A M. Interleukin 1 protects against 1-beta-D-arabinofuranosylcytosine-induced alopecia in the newborn rat model. Cancer Res 1991; 51(12):3329-30.
6. Balsari A L, Morelli D, Menard S, Veronesi U, Colnaghi M I. Protection against doxorubicin-induced alopecia in rats by liposome-entrapped monoclonal antibodies. FASEB J 1994; 8(2):226-230.
7. Paus R, Handjiski B, Eichmuller S, Czametzki B M. Chemotherapy-induced alopecia in mice—induction by cyclophosphamide, inhibition by cyclosporine-A, and modulation by dexamethasone. Am J Pathol 1994: 144(4):719-734.
8. Hussein A M, Stuart A, Peters W P. Protection against chemotherapy-induced alopecia by cyclosporine A in the newborn rat model. Dermatology 1995; 190(3):192-196.
9. Shirai A, Tsunoda H, Tamaoki T, Kamiya T. Topical application of cyclosporin A induces rapid-remodeling of damaged anagen hair follicles produced in cyclophosphamide administered mice. J Dermatol Sci 2001; 27(1):7-13.
10. Hussein A M. Protection against cytosine arabinoside-induced alopecia by minoxidil in a rat animal model. Int J Dermatol 1995; 34(7):470-3.
11. Jimenez J J, Yunis A A. Protection from 1-beta-D-arabinofuranosylcytosine-induced alopecia by epidermal growth factor and fibroblast growth factor in the rat model. Cancer Res 1992; 52(2):413-5.
12. Danilenko D M, Ring B D, Yanagihara D, Benson W, Wiemann B, Starnes C O, et al. Keratinocyte growth factor is an important endogenous mediator of hair follicle growth, development, and differentiation: normalization of the nu/nu follicular differentiation defect and amelioration of chemotherapy-induced alopecia. Am J Pathol 1995; 147(1):145-54.
13. Botchkarev V A, Komarova E A, Siebenhaar F, Botchkareva N V, Komarov P G, Maurer M, et al. p53 is essential for chemotherapy-induced hair loss. Cancer Res 2000; 60(18):5002-5006.
14. Davis S T, Benson B G, Bramson H N, Chapman D E, Dickerson S H, Dold K M, et al. Prevention of chemotherapy-induced alopecia in rats by CDK inhibitors. Science 2001; 291(5501):134-137.
15. Davis S T, Benson B G, Bramson H N, Chapman D E, Dickerson S H, Dold K M, et al. Retraction. Science 2002; 298(5602):2327.
16. Tsuda T, Ohmori Y, Muramatsu H. Hosaka Y, Takiguchi K. Saitoh F, et al. Inhibitory effect of M50054, a novel inhibitor of apoptosis, on anti-Fas-antibody-induced hepatitis and chemotherapy-induced alopecia. Eur J Pharmacol 2001; 433(1):37-45.
17. Jimenez J J, Haung H S, Yunis A A. Treatment with ImuVert/N-acetylcysteine protects rats from cyclophosphamide/cytarabine-induced alopecia. Cancer Invest 1992; 10(4):271-6.
18. Olsen E A (editor): Disorders of Hair Growth: Diagnosis and Treatment, New York, McGraw-Hill, 2003, and references cited therein.

1. A method of inhibiting hair growth, the method comprising administering to a subject in need thereof, a safe and effective amount of a compound of formula (I), or a pharmaceutically acceptable salt thereof, 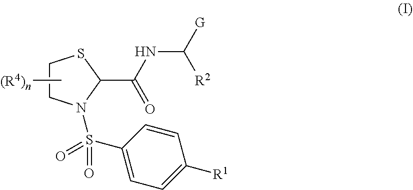
wherein:
G is 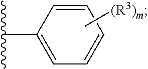
R¹ is tert-butyl or phenyl, wherein the phenyl is optionally substituted with 1-3 R^{1a};
R^{1a}, at each occurrence, is independently C₁₋₄alkyl, C₁₋₄fluoroalkyl, halogen, cyano, —OC₁₋₄alkyl, or —OC₁₋₄fluoroalkyl;
R² is —C₁₋₄alkylene-H, —C₁₋₄alkylene-OC₁₋₄alkyl; —C₁₋₄alkylene-OC(O)C₁₋₄alkyl, or —C₁₋₄alkylene-OC(O)CH(NH₂)R^{2a};
R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄alkylene-OH, or —C₁₋₄alkylene-OC₁₋₄alkyl;
R³, at each occurrence, is independently C₁₋₄alkyl, C₁₋₄fluoroalkyl, halogen, cyano, —OC₁₋₄alkyl, or —OC₁₋₄fluoroalkyl;
R⁴, at each occurrence, is independently C₁₋₄alkyl or C₁₋₄fluoroalkyl;
m is 0, 1, 2, 3, 4, or 5; and
n is 0, 1, 2, 3, or 4.

2. A method of treating a condition, the method comprising administering to a subject in need thereof, a safe and effective amount of a compound of formula (I), or a pharmaceutically acceptable salt thereof, wherein the condition is selected from at least one of hirsutism, hypertrichosis, unwanted hair, chemotherapy-related hair loss, radiation-related hair loss, and a combination thereof, 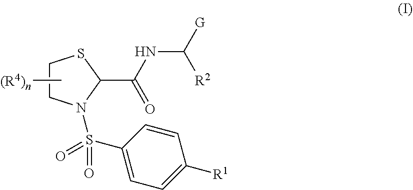
wherein:
G is 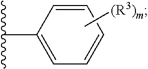
R¹ is tert-butyl or phenyl, wherein the phenyl is optionally substituted with 1-3 R^{1a};
R^{1a}, at each occurrence, is independently C₁₋₄alkyl, C₁₋₄fluoroalkyl, halogen, cyano, —OC₁₋₄alkyl, or —OC₁₋₄fluoroalkyl;
R² is —C₁₋₄alkylene-OH, —C₁₋₄alkylene-OC₁₋₄alkyl; —C₁₋₄alkylene-OC(O)C₁₋₄alkyl, or —C₁₋₄ alkylene-OC(O)CH(NH₂)R^{2a};
R^{2a} is hydrogen, C₁₋₄alkyl, C₁₋₄fluoroalkyl, C₃₋₆cycloalkyl, —C₁₋₄alkylene-C₃₋₆cycloalkyl, —C₁₋₄ alkylene-OH, or —C₁₋₄alkylene-OC₁₋₄alkyl;
R³, at each occurrence, is independently C₁₋₄alkyl, C₁₋₄fluoroalkyl, halogen, cyano, —OC₁₋₄alkyl, or —OC₁₋₄fluoroalkyl;
R⁴, at each occurrence, is independently C₁₋₄alkyl or C₁₋₄fluoroalkyl;
m is 0, 1, 2, 3, 4, or 5; and
n is 0, 1, 2, 3, or 4.

3. The method of any of claims 1-2, wherein R¹ is phenyl.

4. The method of any of claims 1-3, wherein R² is —C₁₋₄alkylene-OH.

5. The method of claim 4, wherein R² is —CH₂CH₂OH.

6. The method of any of claims 1-3, wherein R² is —C₁₋₄alkylene-OC(O)CH(NH₂)R^{2a}.

7. The method of claim 6, wherein R² is —CH₂CH₂—OC(O)CH(NH₂)R^{2a}.

8. The method of claim 6, wherein R² is —C₁₋₄alkylene-OC(O)CH(NH₂)CH(CH₃)₂.

9. The method of claim 6, wherein R² is —CH₂CH₂—OC(O)CH(NH₂)CH(CH₃)₂.

10. The method of any of claims 1-9, wherein R³ is fluoro, chloro, CH₃, or OCH₃; and m is 0, 1, or 2.

11. The method of any of claims 1-10, wherein G is

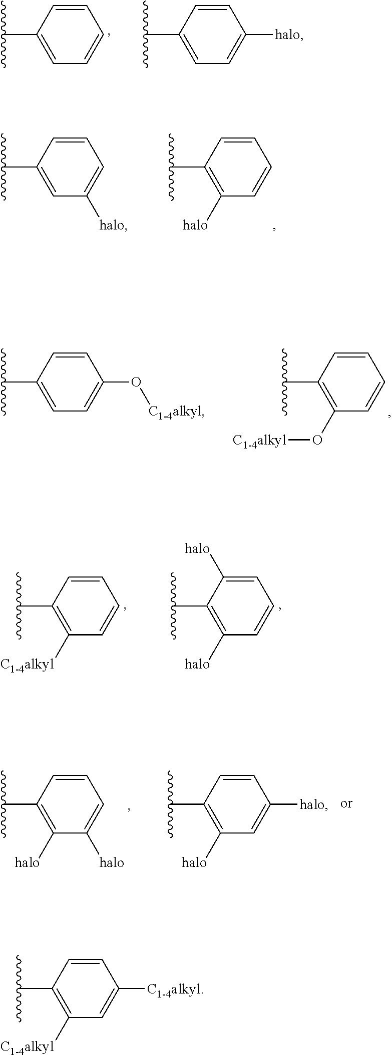

12. The method of claim 11, wherein G is

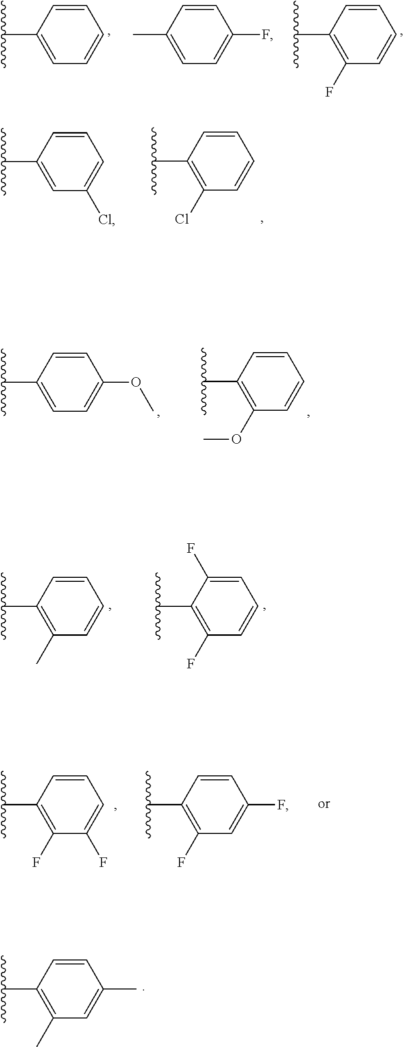

13. The method of any of claims 1-12, wherein n is 0.

14. The method of any of claims 1-13, wherein the compound of formula (I) has formula (I-A)

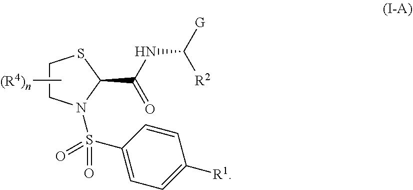

15. The method of any of claims 1-2, wherein the compound of formula (I) is

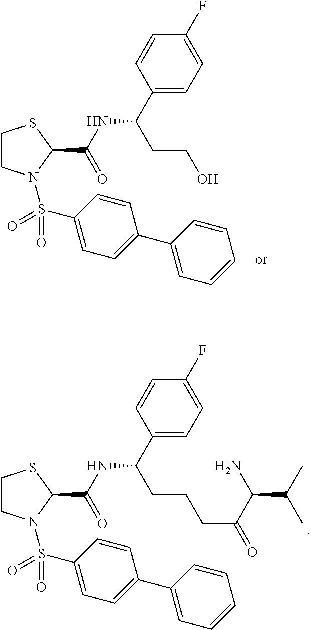 [SMILES: O=C(N[C@@H](CCO)c1ccc(F)cc1)[C@@H]1SCCN1S(=O)(=O)c1ccc(-c2ccccc2)cc1 ; CC(C)[C@H](N)C(=O)CCC[C@H](NC(=O)[C@@H]1SCCN1S(=O)(=O)c1ccc(-c2ccccc2)cc1)c1ccc(F)cc1]

16. A method of inhibiting hair growth, the method comprising administering to a subject in need thereof, a safe and effective amount of a compound of formula (II), or a pharmaceutically acceptable salt thereof, 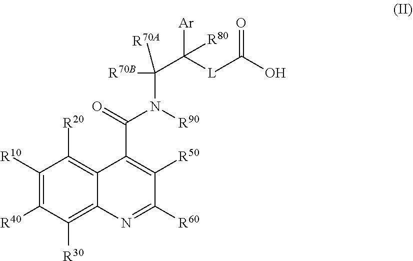
wherein
Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄ alkyl, C₃₋₄cycloalkyl, —OC₁₋₂alkyl, —SC₁₋₂alkyl, C₁₋₄alkyl substituted by 1-3 fluoro, C₃₋₄ cycloalkyl substituted by 1-4 fluoro, —OC₁₋₂alkyl substituted by 1-3 fluoro, —SC₁₋₂alkyl substituted by 1-3 fluoro, or where two substituents of the phenyl or pyridinyl on adjacent ring atoms together form a methylenedioxy or ethylenedioxy;
L is a bond or —X—(CR^{a}R^{b})ₖ—;
X is a bond, CH₂, O, S, S(O), S(O)₂, or N(R);
R is hydrogen or CH₃;
R^{a} and R^{b} are independently hydrogen, fluoro, or CH₃; or R^{a} and R^{b} together with the carbon atom to which they attach form a cyclopropyl;
k is 1, 2, 3, or 4;
R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ are independently hydrogen, halogen, CH₃, CH₂F, CHF₂, or CF₃;
R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ is —NR^{c}R^{d};
R^{c} is hydrogen or C₁₋₃alkyl;
R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} are independently hydrogen or CH₃, or R^{70A} and R^{70B} together with the carbon atom to which they attach form a cyclopropyl;
R⁸⁰ is hydrogen, fluoro, CH₃, CF₃, CH₂CH₃, or OH; and
R⁹⁰ is hydrogen or CH₃.

17. A method of treating a condition, the method comprising administering to a subject in need thereof, a safe and effective amount of a compound of formula (II), or a pharmaceutically acceptable salt thereof, wherein the condition is selected from at least one of hirsutism, hypertrichosis, unwanted hair, chemotherapy-related hair loss, radiation-related hair loss, and a combination thereof, 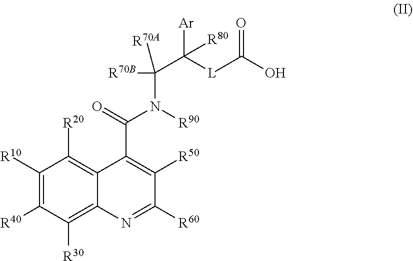
wherein
Ar is phenyl or pyridinyl, wherein the phenyl is unsubstituted or substituted with 1-5 substituents, the pyridinyl is unsubstituted or substituted with 1-2 substituents, and the substituents are independently selected from the group consisting of fluoro, chloro, C₁₋₄ alkyl, C₃₋₄cycloalkyl, —OC₁₋₂alkyl, —SC₁₋₂alkyl, C₁₋₄alkyl substituted by 1-3 fluoro, C₃₋₄ cycloalkyl substituted by 1-4 fluoro, —OC₁₋₂alkyl substituted by 1-3 fluoro, and —SC₁₋₂alkyl substituted by 1-3 fluoro, or where two substituents of the phenyl or pyridinyl on adjacent ring atoms together form a methylenedioxy or ethylenedioxy;
L is a bond or —X—(CR^{a}R^{b})ₖ—;
X is a bond, CH2, O, S, S(O), S(O)₂, or N(R);
R is hydrogen or CH₃;
R^{a} and R^{b} are independently hydrogen, fluoro, or CH₃; or R^{a} and R^{b} together with the carbon atom to which they attach form a cyclopropyl;
k is 1, 2, 3, or 4;
R¹⁰ is halogen, C₁₋₄alkyl, C₁₋₄alkyl substituted by 1-5 fluoro, —OCH₃, —OCH₂F, —OCHF₂, —OCF₃, —SCF₃, —SF₅, —SiCH₃, ethynyl, cyclopropyl, or cyclobutyl, where the cyclopropyl and cyclobutyl are unsubstituted or substituted by 1-4 fluoro;
R²⁰, R³⁰, and R⁴⁰ are independently hydrogen, halogen, CH₃, CH₂F, CHF₂, or CF₃;
R⁵⁰ is halogen, C₁₋₄alkyl, C₁₋₄alkyl substituted by 1-5 fluoro, —OCH₃, —OCH₂F, —OCHF₂, —OCF₃, OH, —SCH₃, —SCF₃, cyano, ethenyl, cyclopropyl, or cyclobutyl, where the cyclopropyl and cyclobutyl are unsubstituted or substituted by 1-4 fluoro;
R⁶⁰ is —NR^{c}R^{d};
R^{c} is hydrogen or C₁₋₃alkyl;
R^{d} is C₁₋₄alkyl, C₃₋₇cycloalkyl, C₁₋₄alkyl substituted by 1-5 fluoro, C₁₋₄alkyl monosubstituted by C₃₋₆cycloalkyl, —OCH₃, —OCF₃, or phenyl, wherein the C₃₋₇cycloalkyl is optionally substituted with 1-4 fluoro; or
R^{c} and R^{d} together with the nitrogen to which they attach form a saturated or partially unsaturated 4- to 8-membered monocyclic or 6- to 10-membered bicyclic heterocyclic ring, the heterocyclic rings optionally containing a ring member selected from the group consisting of O, N, S, SO, and S(O)₂, wherein the heterocyclic rings are optionally substituted with 1-4 substituents independently selected from the group consisting of fluoro, C₁₋₄alkyl, OH, oxo, —OC₁₋₃alkyl, —OCHF₂, —OCF₃, cyano, NH₂, NHCH₃, N(CH₃)₂, C(O)NH₂, C(O)NHCH₃, C(O)N(CH₃)₂, C₁₋₄alkyl substituted with 1-5 fluoro, and C₁₋₄alkyl monosubstituted with OH or —OCH₃;
R^{70A} and R^{70B} are independently hydrogen or CH₃, or R^{70A} and R^{70B} together with the carbon atom to which they attach form a cyclopropyl;
R⁸⁰ is hydrogen, fluoro, CH₃, CF₃, CH₂CH₃, or OH; and
R⁹⁰ is hydrogen or CH₃.

18. The method of claim 16 or 17, wherein,
Ar is phenyl, wherein the phenyl is unsubstituted or substituted with 1-4 fluoro, or substituted with 1-3 substituents independently selected from the group consisting of fluoro, chloro, CH₃, CHF₂, CF₃, OCH₃, OCHF₂, and OCF₃;
L is a bond or —(CR^{a}R^{b})ₖ—
k is 1, 2, or 3;
R¹⁰ is bromo or ethynyl;
R²⁰, R³⁰, and R⁴⁰ are hydrogen;
R⁵⁰ is chloro or CH₃;
R^{c} is hydrogen or CH₃;
R^{d} is C₁₋₄alkyl, C₁₋₄alkyl substituted by 1-3 fluoro, or C₁₋₄alkyl monosubstituted by phenyl; or
R^{c} and R^{d} together with the nitrogen to which they attach form a saturated or partially unsaturated 5- to 7-membered monocyclic or 7- to 10-membered bicyclic heterocyclic ring, the heterocyclic rings optionally containing a ring member selected from the group consisting of O, N, and S, wherein the heterocyclic rings are optionally substituted with 1-2 substituents independently selected from the group consisting of CH₃, CH₂CH₃, CH(CH₃)₂, CHF₂, CF₃, CH₂CHF₂, and CH₂CF₃, and optionally substituted with 1-4 fluoro; and
R^{70A}, R^{70B}, R⁸⁰, and R⁹⁰ are hydrogen.

19. The method of claim 18, wherein,
Ar is phenyl, wherein the phenyl is unsubstituted or substituted with 1-3 substituents independently selected from the group consisting of fluoro, chloro, CH₃, CF₃, OCH₃, OCHF₂, and OCF₃;
L is —CH₂CH₂—;
R¹⁰ is bromo;
R⁵⁰ is CH₃;
R⁶⁰ is 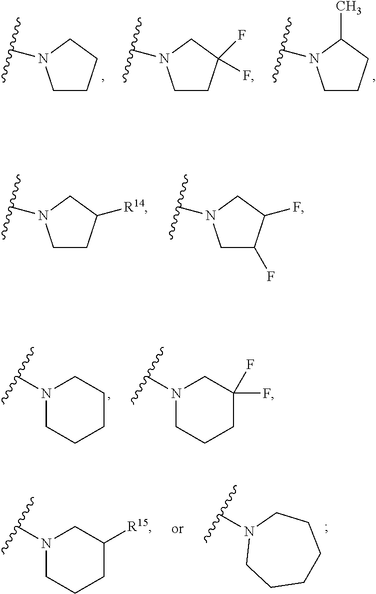
R¹⁴ is fluoro or CH₃; and
R¹⁵ is fluoro, CH₃, or CH₂CH₃.

20. The method of claim 19, wherein the compound of formula (II) is

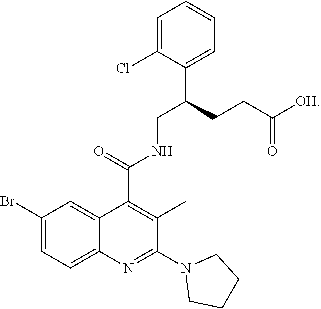 [SMILES: Cc1c(N2CCCC2)nc2ccc(Br)cc2c1C(=O)NC[C@H](CCC(=O)O)c1ccccc1Cl]

21. The method of any of claims 1, 3-16, or 18-20, wherein inhibiting hair growth includes slowing hair growth.

22. The method of any of claims 1, 3-16, or 18-20, wherein inhibiting hair growth includes stopping hair growth.

23. The method of any of claims 1, 3-16, or 18-20, wherein inhibiting hair growth includes preventing hair regrowth after hair removal.

24. The method of any of claims 1, 3-16, or 18-20, wherein the inhibition of hair growth treats at least one of hirsutism, hypertrichosis, or unwanted hair, and/or prevents or limits at least one of regrowth after hair removal, chemotherapy-related hair loss, or radiation-related hair loss, and a combination thereof.

25. The method of any of claims 2-15 or 17-20, wherein the condition is hirsutism.

26. The method of any of claims 2-15 or 17-20, wherein the condition is hypertrichosis.

27. The method of any of claims 2-15 or 17-20, wherein the condition is unwanted hair.

28. The method of any of claims 2-15 or 17-20, wherein the condition is chemotherapy-related hair loss.

29. The method of any of claims 2-15 or 17-20, wherein the condition is radiation-related hair loss.

30. The method of claim 28 or 29, wherein the compound of formula (I) or (II), or pharmaceutically acceptable salt thereof, is administered prior to chemotherapy or radiation, and inhibits hair growth and renders the subject less susceptible to chemotherapy-related hair loss or radiation-related hair loss.
